(19) United States
(12) Patent Application Publication
(10) Pub. No.: US 2017/0311816 A1
(43) Pub. Date: Nov. 2, 2017

(54) BODY METRIC MEASUREMENT SYSTEMS, DEVICES, AND METHODS

(71) Applicants: Perry Jeter, Columbus, OH (US); Lloyd Tripp, Dayton, OH (US); Adam Reener, Columbus, OH (US); Bob Lee, Beavercreek, OH (US)

(72) Inventors: Perry Jeter, Columbus, OH (US); Lloyd Tripp, Dayton, OH (US); Adam Reener, Columbus, OH (US); Bob Lee, Beavercreek, OH (US)

(21) Appl. No.: 15/583,972

(22) Filed: May 1, 2017

Related U.S. Application Data
(60) Provisional application No. 62/329,283, filed on Apr. 29, 2016.

(51) Int. Cl. A61B 5/0205; A61B 5/00; A61B 5/1455; A61B 5/11; A61B 5/024; A61B 90/00

(52) U.S. Cl. CPC A61B 5/0205; A61B 5/14552; A61B 5/1123; A61B 5/6815; A61B 2090/064; A61B 5/02433; A61B 2562/0238

(57) ABSTRACT

A system for providing body metric measurements is provided, the system comprising: a light source providing a red light and an infrared light; an optical detector configured to receive reflected optical signals; force sensor, wherein the force sensor is integrated into a wearable device; and one or more processor configured to automatically transform signals indicative of the reflected optical signals and the force into body metric measurements.

### GOVERNMENT LICENSE RIGHTS

This invention was made with government support under United States Air Force Cooperative Research and Development Agreement No. 16-076-RH-03CRD. The government has certain rights in the invention.

### BACKGROUND

The present specification generally relates to systems, devices, and methods for measuring body metrics and, more specifically, to systems, devices, and methods for measuring body metrics using optical detectors and force sensors integrated with wearable devices.

Unless otherwise indicated herein, the materials described in this section are not prior art to the claims in this application and are not admitted to be prior art by inclusion in this section.

Generally, body metric measurements are performed and tracked by health care professionals, e.g., doctors, nurses, or hospitals. Thus, the body metrics are generally interpreted by the health care professionals to diagnose and treat certain conditions associated with the body metrics. Some patients can find the collection of body metrics to be invasive or inconvenient. Also, the data sets of body metrics form an incomplete picture of the of the diagnosed person and populations of people. Moreover, the data remains under the control of the health care professionals.

Accordingly, a need exists for alternative systems, devices, and methods for measuring body metrics.

### SUMMARY

In one embodiment, a method for providing body metric measurements can include detecting reflected optical signals from a body of a user with an optical detector. The reflected optical signals can include red light and infrared light. A force applied to the user can be detected with a force sensor. The force sensor can be integrated into a wearable device. Signals indicative of the reflected optical signals and the force can be transformed, automatically with one or more processors, into body metric measurements.

In another embodiment, a method for providing body metric measurements is provided, the method comprising: detecting reflected optical signals from a body of a user with an optical detector, wherein the reflected optical signals comprise red light and infrared light; detecting a force applied to the user with a force sensor, wherein the force sensor is integrated into a wearable device; and transforming, automatically with one or more processors, signals indicative of the reflected optical signals and the force into body metric measurements.

In one embodiment, a system for providing body metric measurements is provided, the system comprising: a light source providing a red light and an infrared light; an optical detector configured to receive reflected optical signals; force sensor, wherein the force sensor is integrated into a wearable device; and one or more processor configured to automatically transform signals indicative of the reflected optical signals and the force into body metric measurements.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a system for measuring body metric according to one or more embodiments shown and described herein.

FIG. 2 schematically depicts a system for measuring body metric according to one or more embodiments shown and described herein.

FIG. 3 schematically depicts a system for measuring body metric according to one or more embodiments shown and described herein.

FIG. 4 graphically depicts output signals provided by the system of FIGS. 2 and 3 according to one or more embodiments shown and described herein.

FIG. 5 schematically depicts a race system utilizing a system for measuring body metrics.

FIG. 6 schematically depicts an application for a pit crew utilizing a system for measuring body metrics.

FIG. 7 illustrates a G-profile to which a subject was exposed.

FIG. 8 illustrates a G-profile to which a subject was exposed.

FIG. 9 illustrates a G-profile to which a subject was exposed.

FIG. 10 illustrates a G-profile to which a subject was exposed.

FIG. 11 illustrates a G-profile to which a subject was exposed.

FIG. 12 illustrates a comparison of the ear plug heart rate sensor and a wrist-mounted heart rate sensor during exposure of Subject 3 to the various G-profiles.

FIG. 13 illustrates a comparison of the ear plug heart rate sensor and a wrist-mounted heart rate sensor during exposure of Subject 5 to the various G-profiles.

FIG. 14 illustrates the altitude profile to which the subjects were exposed.

FIG. 15 illustrates a comparison of the ear plug heart rate sensor and a wrist-mounted heart rate sensor during exposure of Subject 1 to hypoxia at a simulated altitude of 17,500 ft.

FIG. 16 illustrates a comparison of the ear plug heart rate sensor and a wrist-mounted heart rate sensor during exposure of Subject 2 to hypoxia at a simulated altitude of 17,500 ft.

FIG. 17 illustrates a comparison of the ear plug heart rate sensor and a wrist-mounted heart rate sensor during exposure of Subject 3 to hypoxia at a simulated altitude of 17,500 ft.

FIG. 18 illustrates a comparison of the ear plug heart rate sensor and a wrist-mounted heart rate sensor during exposure of Subject 4 to hypoxia at a simulated altitude of 17,500 ft.

FIG. 19 illustrates a comparison of the ear plug heart rate sensor and a wrist-mounted heart rate sensor during exposure of Subject 5 to hypoxia at a simulated altitude of 17,500 ft.

FIG. 20 illustrates a comparison of the heart rate and photoplethysmogram (“PPG”) versus time of the ear plug heart rate sensor.

### DETAILED DESCRIPTION

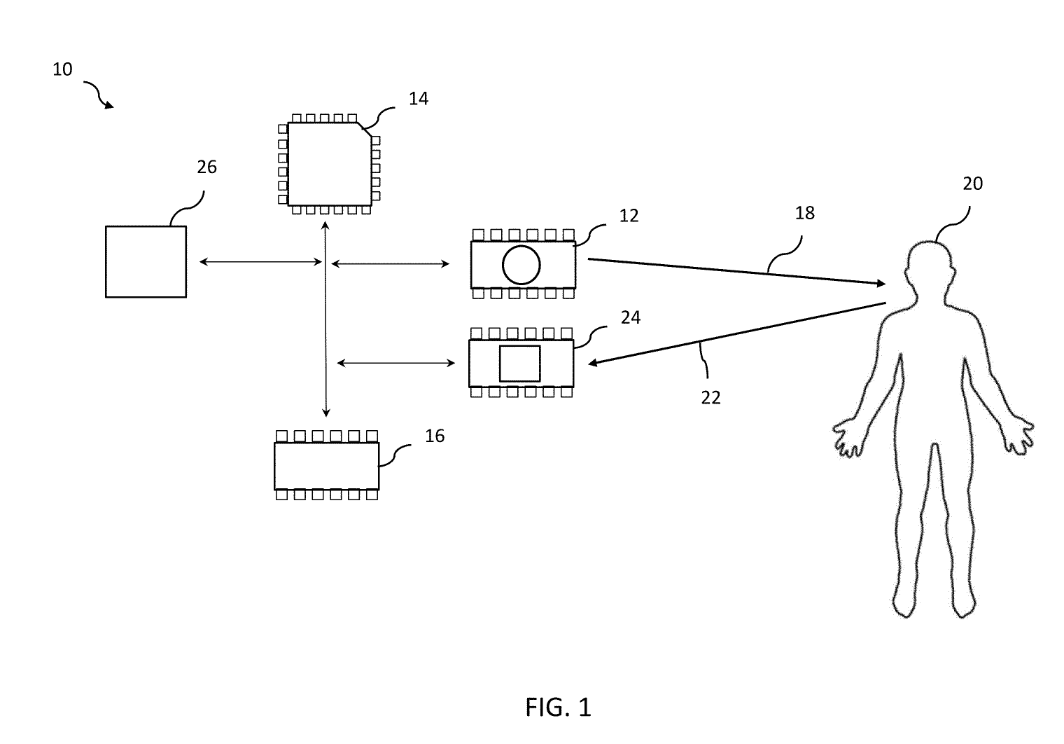

FIG. 1 generally depicts one embodiment of a system for measuring body metrics. The system generally comprises a light source for generating one or wavelengths of optical signals, an optical detector for detecting back reflected optical signals from a user, and a force sensor for detecting force or shock from a user. The system can be configured to correlate the detected optical signals and force/shock to one or more body metrics of the user. Various embodiments of the system and the operation of the system will be described in more detail herein.

Referring now to FIG. 1, an embodiment of a system 10 for measuring body metrics is schematically depicted. The system 10 can comprise a light source 12 communicatively coupled (generally depicted as double arrowed lines) to one or more processors 14 and memory 16. As used herein, the phrase “communicatively coupled” can mean that components are capable of exchanging data signals with one another such as, for example, electrical signals via conductive medium, electromagnetic signals via air, optical signals via optical waveguides, and the like.

The light source 12 can be configured to emit one or more optical signals 18 of a desired wavelength. The light source 12 can comprise one or more light emitting diodes (LED or OLED), or any other electrical device suitable for emitting the desired optical signals. The desired wavelength can be any wavelength that can interact with the body of a user 20 to generate one or more reflected optical signal 22 indicative of a body metric of the user 20 such as, for example, pulse oximetry, heart rate, or the like. For example, the one or more optical signals 18 can comprise a first optical signal of red light, i.e., between about 620 nm and about 750 nm such as, for example, between about 650 nm and about 670 nm, in one embodiment. Alternatively or additionally, the one or more optical signals 18 can comprise a second optical signal of infrared light, i.e., between about 750 nm and about 1 mm such as, for example, between about 870 nm and about 900 nm, in one embodiment. It is noted that the term “signal,” as used herein, can mean a waveform (e.g., electrical, optical, magnetic, or electromagnetic), such as DC, AC, sinusoidal-wave, triangular-wave, square-wave, and the like, capable of traveling through a medium. It is furthermore noted that the term “optical” or “light” can refer to various wavelengths of the electromagnetic spectrum such as, but not limited to, wavelengths in the ultraviolet (UV), infrared (IR), and visible portions of the electromagnetic spectrum.

The one or more processors 14 can comprise any device capable of executing machine readable instructions. Accordingly, each of the one or more processors 14 can be a controller, an integrated circuit, a microchip, or any other device capable of implementing logic. Specific examples of one of the processors 14 can include a microprocessor, a microcontroller, a system on a chip, a signal processor, a touch screen controller, a baseband controller, graphics processor, application processor, image processor, or the like.

The memory 16 described herein may be RAM, ROM, a flash memory, a hard drive, or any device capable of storing machine readable instructions. Additionally, it is noted that the functions and processes described herein can be provided as machine readable instructions stored on memory 16 and executed by the one or more processors 14. The machine readable instructions can be provided in any programming language of any generation (e.g., 1GL, 2GL, 3GL, 4GL, or 5GL) such as, e.g., machine language that may be directly executed by the processor, or assembly language, object-oriented programming (OOP), scripting languages, microcode, etc., that may be compiled or assembled into machine readable instructions and stored on a machine readable medium. Alternatively, the functions, modules, and processes described herein may be written in a hardware description language (HDL), such as logic implemented via either a field-programmable gate array (FPGA) configuration or an application-specific integrated circuit (ASIC), and their equivalents. Accordingly, the functions and processes described herein may be implemented in any conventional computer programming language, as pre-programmed hardware elements, or as a combination of hardware and software components.

Referring still to FIG. 1, the system 10 can comprise an optical detector 24 communicatively coupled to the one or more processors 14 for detecting the one or more reflected optical signals 22 and encoding the detected signals into an electrical signal. The optical detector 24 can comprise a photosensor, photodetector, photodiode, or the like. Accordingly, the optical detector 24 can be tuned to detect substantially the same wavelength as the desired optical signals, or any wavelengths that the light source 12 is configured to transmit.

According to the embodiments described herein, the system 10 can comprise a force sensor 26 communicatively coupled to the one or more processors 14 for detecting a force or shock acting upon the user 20 and encoding the detected force into an electrical signal. In some embodiments, the force sensor 26 can be configured to directly measure the force or shock. Accordingly, the force sensor 26 can comprise load cell configured to measure, for example, tension, compression, shear, strain, or a combination thereof. Alternatively or additionally, the force sensor 26 can be configured to indirectly measure the force or shock by detecting parameters that can be correlated to force. For example, the force sensor 26 can be configured to detect motion, e.g., linear motion or directional motion, which can be correlated to impact force, rotational motion which can be correlated to whiplash, combinations thereof, or the like. Accordingly, the force sensor 26 can comprise linear, angular, or multi-axis positional sensors such as, for example, an accelerometer, an accelerometer, a gyroscope, a magnetometer, or combinations thereof.

In the embodiments described herein, the light source 12, the one or more processors 14, memory 16, optical detector 24, and the force sensor 26 can be provided within a unitary device. However, it is noted that the light source 12, the one or more processors 14, memory 16, optical detector 24, and the force sensor 26 can be discrete components provided in multiple devices and communicatively coupled with one another without departing from the scope of the present disclosure. Accordingly, the system 10 can be integrated within one or more articles such as, but not limited to, clothing, accessories (e.g., belts, watches, shoes, hats, etc.), or any other wearable device.

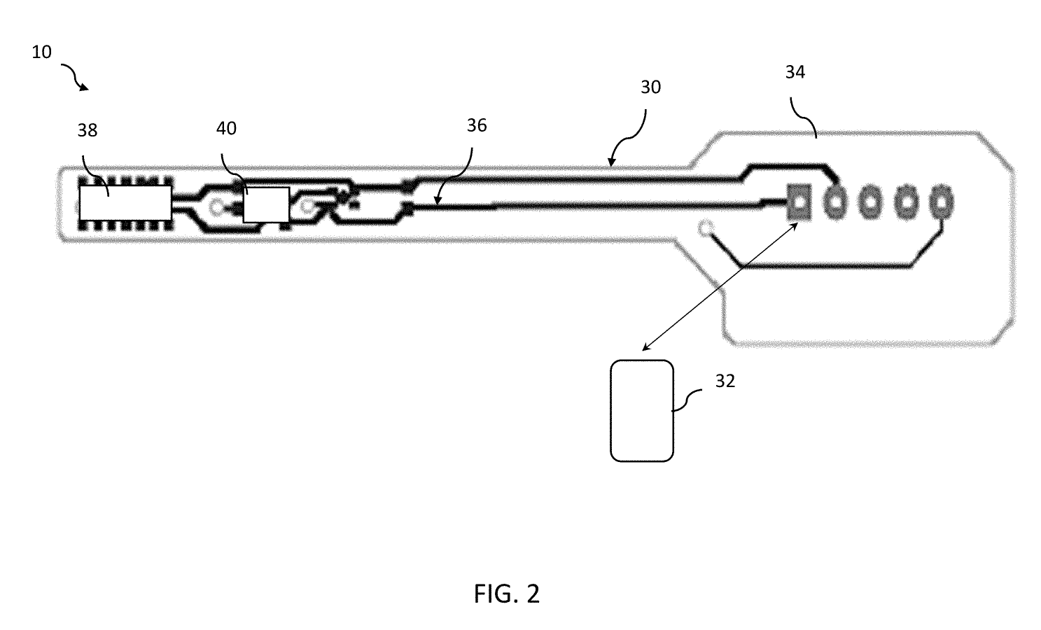

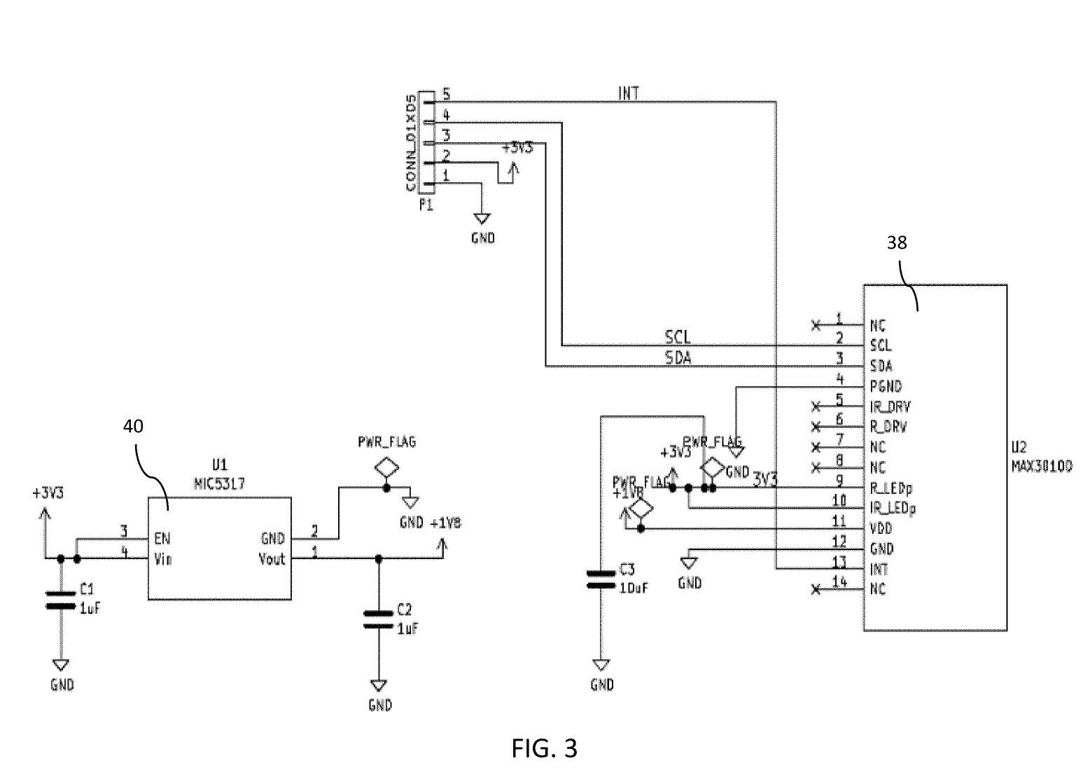

Referring collectively to FIGS. 1, 2, and 3, in some embodiments, the system 10 can be provided as a sensing component 30 communicatively coupled to a connected device 32. Accordingly, the sensing component 30, the connected device 32 or both can be configured to send and/or receive data signals via any wired or wireless communication protocol. For example, the sensing component 30 and the connected device 32 can be communicatively coupled via wired interfaces or computer buses such as, for example, USB, FIREWIRE, CAN Bus, LIN Bus, or the like. Alternatively or additionally, the sensing component 30 and the connected device 32 can be communicatively coupled via a wireless interface such as, for example, a personal area network. Suitable personal area networks can comprise wireless technologies such as, for example, IrDA, BLUETOOTH, Wireless USB, Z-WAVE, ZIGBEE, or the like.

The sensing component 30 can comprise a subset of the components of the system 10, and the connected device can be configured to comprise a subset of the components of the system 10. The sensing component 30 can comprise a printed circuit board 34 comprising a plurality of conductive traces 36 for communicatively coupling a sensing system chip 38 to the connected device 32 and a power regulation chip 40. The sensing system chip 38 can be configured as a system on a chip. For example, the sensing system chip 38 can comprise the light source 12, the one or more processors 14, memory 16, and the optical detector 24. One example of a suitable system on a chip for use as the sensing system chip 38 is the MAX30100 by Maxim Integrated of San Jose, Calif., U.S.A. One example of a suitable component for use as the power regulation chip 40 is the MIC5317 by Micrel Inc. of San Jose, Calif., U.S.A.

In some embodiments, it can be desired to provide the sensing component 30 in a relatively small form factor. For example, the sensing component 30 can be sized to position the sensing system chip 38 within the ear of the user 20 (e.g., integrated into an ear plug, ear buds, headphones, or headset). In order to maintain the relatively small size, a portion of the one or more processors 14 and memory 16 can be provided on the sensing component 30 and a portion of the one or more processors 14 and memory 16 can be provided on the connected device 32. For example, the sensing system chip 38 can comprise a portion of the one or more processors 14 and memory 16 suitable to perform detection, signal processing functions, and output of signals indicative of the detected signals. The connected device 32 can comprise the portion of the one or more processors 14 and memory 16 suitable for controlling the operation of the sensing system chip 38 and for transforming the output of signals of the sensing system chip 38 into body metrics of the user 20. Alternatively or additionally, the connected device can be configured to power the sensing component 30. The connected component 32 can comprise a smart phone, a mobile phone, a tablet, a laptop computer, desktop computer, vehicle, or any specialized machine having communication and processing capability. Accordingly, the system 10 can be provided in a modular fashion that can allow sensors to be scaled for integration within any desired wearable device.

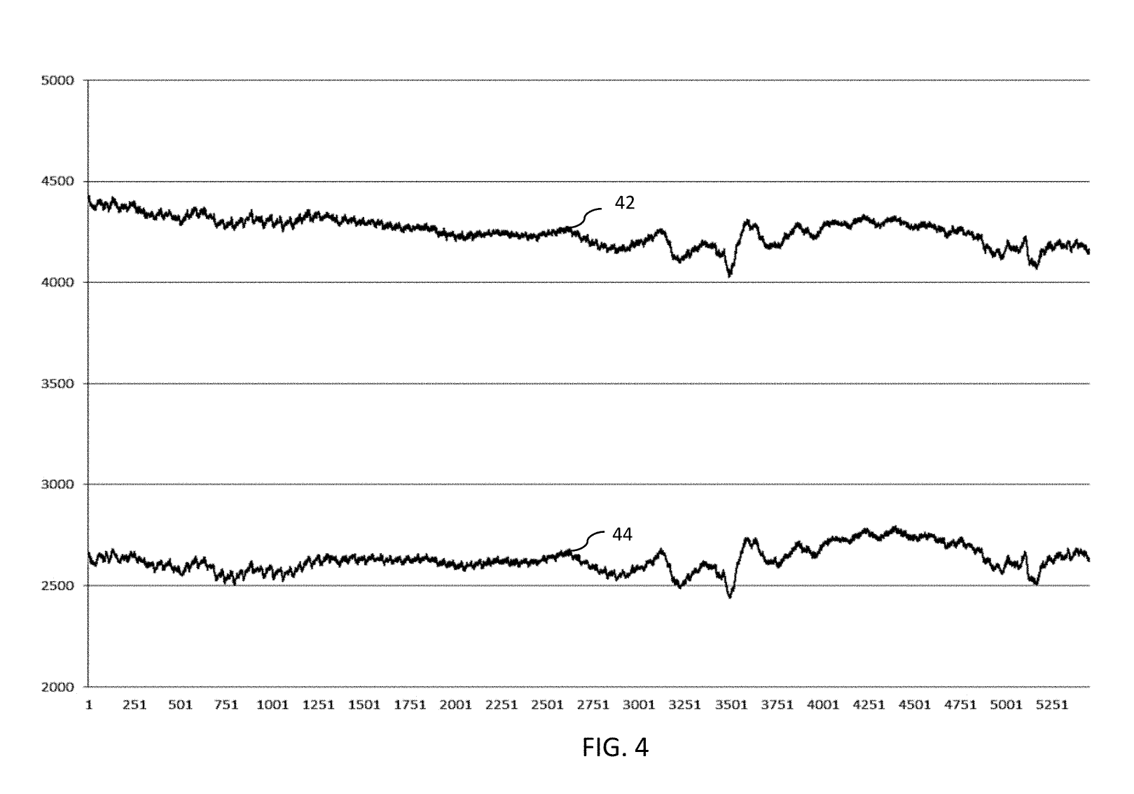

Referring collectively to FIGS. 1 and 4, the one or more reflected optical signals 22 detected by the optical detector 24 can be transformed into a first output signal 42 and a second output signal 44. Each of the first output signal 42 and the second output signal 44 can indicate the intensity or power level of a wavelength of the one or more reflected optical signals 22. Accordingly, the amount of absorption of the one or more optical signals 18 by the user 20 can be inferred. For example, the one or more reflected optical signals 22 were detected using the MAX30100 and the first output signal 42 corresponding to red light and the second output signal 44 corresponding to infrared light were output. It is noted that the MAX3100 further comprises a temperature sensor that can be used to supplement body metric detection as described herein.

The one or more processors 14 can implement algorithms to transform the first output signal 42 and the second output signal 44 to body metrics of the user 20 such as, for example, heart rate and blood oxygen saturation. Body metrics may additional include sleep and rest accumulated by a user, such that one can monitor the user's sleeping and resting habits. Specifically, the algorithms can be executed to perform the function of a pulse oximeter. The pulse oximeter functions can be used to calculate blood oxygen saturation based on the different rates that oxygenated hemoglobin and reduced hemoglobin absorb different wavelengths of light. Generally, the user's 20 absorption of infrared light can be less sensitive to blood oxygen saturation levels than absorption of red wavelengths. Accordingly, the intensity of infrared light in the one or more reflected optical signals 22 after passing through vascular tissue can be used as a constant against which to measure the intensity of the red light in the one or more reflected optical signals 22 after passing through the same vascular tissue. Pulse rate can be calculated from the timing of the relative rise and fall of the one or more reflected optical signals 22 at each wavelength.

As is noted above, the system 10 can be provided in a modular fashion that can allow various components to be integrated within any desired wearable device. Various non-limiting embodiments of the system 10 are provided below. In one embodiment, the light source 12 and the optical detector 24 can be located in a wearable for a pilot (e.g., an ear piece) and the force sensor 26 can be collocated or located in a different wearable (e.g., helmet). Accordingly, the system 10 can be configured for use with aviators to detect G-force induced loss of consciousness (GLOC) or altered level of consciousness (ALOC). For example, output based upon the detected parameters can be communicated to a device in the aircraft that can process the signals and communicate alerts to other aircraft or ground crew. Thus, the system 10 can be used to alert the pilot, ground control, pilot's wingman, a record keeping device, or the like of an GLOC, ALOC, or sleep condition. In another embodiment, the light source 12, the optical detector 24, the force sensor 26 can be located in an earbud having speakers. Accordingly, the system 10 can be used as a training aid that monitors body metrics while the user is being active. Body metrics include, but are not limited to, heart rate, aerobic fitness, speed, pace, cadence, distance and calories burned. Body metrics may include sleep and/or rest.

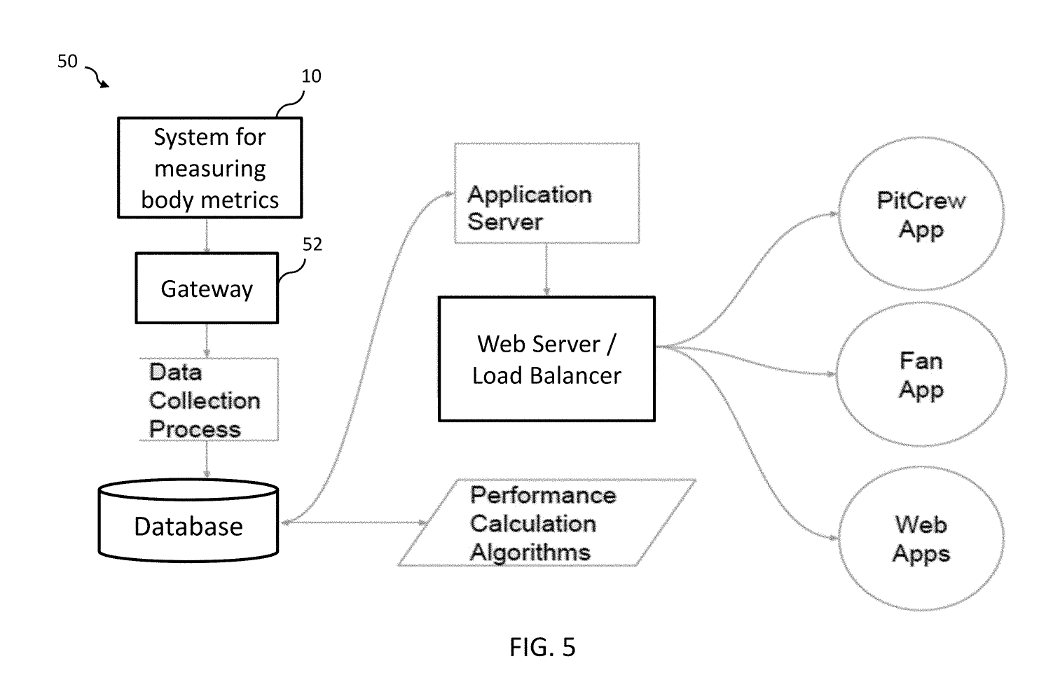

Referring collectively to FIGS. 1, 4, 5, and 6, in one embodiment, the light source 12 and the optical detector 24 can be located in a wearable for a race car driver (e.g., an ear piece) and the force sensor 26 can be collocated or located in a different wearable (e.g., racing helmet). Thus, pit crews or fans can monitor body metrics and force applied to the driver throughout the race. For example, the body metrics and force can be communicated to an application for a computing device that is communicatively coupled to the wearable such, as, for example, a smart phone or tablet. An example race system 50 is depicted in FIG. 5. The race system 50 can comprise the system 10, which can be configured for use with the race system 50. In one embodiment, the system 10 can further comprise a communication component for providing wireless communication. The communication component can comprise a wireless transceiver configured to communicate with a wireless gateway 52. Suitable examples of communication systems include the SX1272 Transceiver, the SX1276 Transceiver, and the SX1301 Concentrator, which incorporate LoRa™, by Semtech Corporation of Camarillo, Calif., U.S.A.

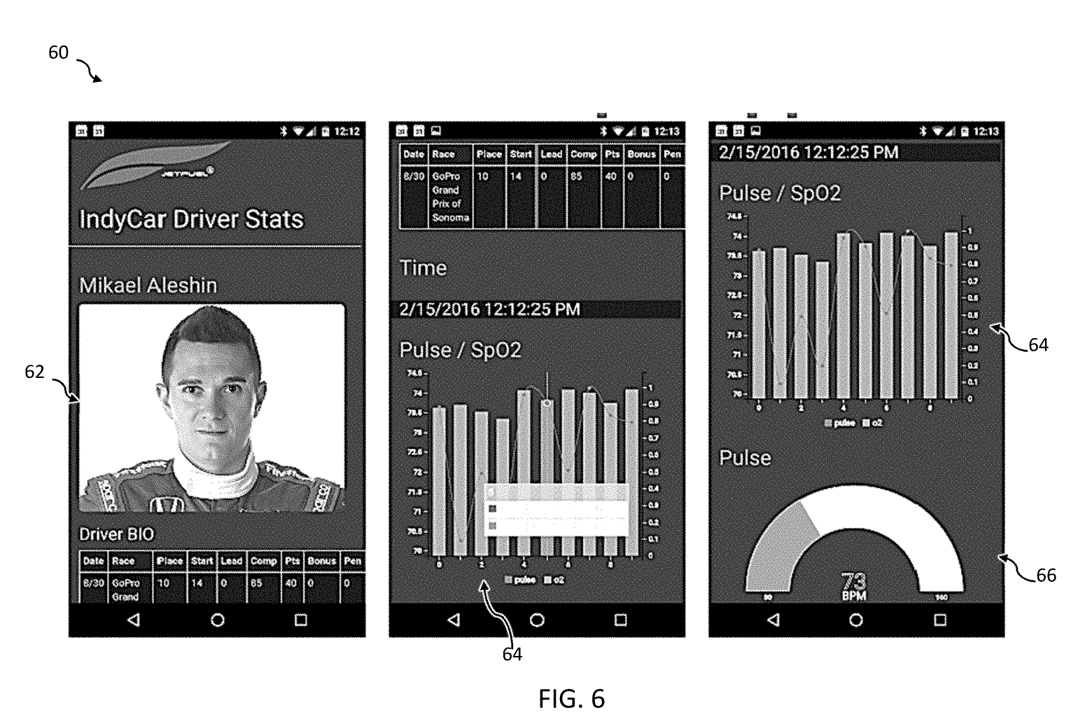

An example application 60 for a pit crew is schematically depicted in FIG. 6. The application 60 for the pit crew can provide the ability to follow a single driver associated with the pit crew. The pit crew can be provided with confidential or restricted information related to the associated driver such as, but not limited to, raw data for logging, real time charts, or the like. Specifically, the application 60 can comprise biographical objects 62 that provide details regarding an associated driver such as, for example, driver image, race history, age, or the like. The application 60 can further comprise trend objects 64 that can be configured to graphically depict body metric and force data over a time period such as, for example, a prior time period, or a time range including the latest detection period, or the like. In some embodiments, the trend objects 64 can be configured as controls that can receive input. For example, the trend objects 64 can respond to a hover input by providing numerical details of the location of the trend object 64 that receives the hover input, i.e., precise body metric data (pulse or oximetry) at a selected time. The application 60 can further comprise real time objects 66 that can be configured to graphically depict body metric and force data at the most recent time of detection, i.e., in real time accounting for communication and detection delays. Moreover, the application 60 can be configured to provide customizable alerts on excursions of certain body metrics from predefined or normal ranges.

The application for the fans can provide the ability to follow a multiple drivers associated with the race event. The fans can be provided with general information such as, but not limited to, information of the state of the driver. The general information can be provided in the form of graphical objects such as, for example, chart graphics, race track graphics, or the like. Accordingly, the embodiments provided herein provide a technological framework that enables the real time monitoring of athlete body metric data. The body metric data can be used to support both athlete performance improvement and fan engagement.

Referring collectively to FIGS. 1 and 4, in another embodiment, the light source 12, the optical detector 24, the force sensor 26 can be located in one or more wearables for a football player such as, for example, in an ear, on a head (e.g., forehead, temples, etc.), in a chin strap, in a mouth guard, or in protective pads. Thus, medical professionals or fans can monitor body metrics and force applied to the football player during in game action and collisions. The force sensor 26 can measure impact forces for concussion, while the optical detector 24 measures other metrics (e.g., blood saturation). Moreover, body metrics such as, for example, core temperature, respirations, resting heart rate, or the like can be measured. Body metrics may also include sleep and/or rest, which may also be measured. In another embodiment, the light source 12, the optical detector 24, the force sensor 26 can be located in an ear guard for wrestlers and configured to perform ear or head readings. In another embodiment, the light source 12, the optical detector 24, the force sensor 26 can be located in an ear or glove of a boxer and configured to perform ear, hand, or wrist readings.

In one embodiment, the light source 12, the optical detector 24, and the force sensor 26 can be located in swimsuits or swimming caps of swimmers. In further embodiments, the light source 12, the optical detector 24, and the force sensor 26 can be located in sports helmet or any other protective helmet and configured to perform forehead or temple readings. In another embodiment, the light source 12, the optical detector 24, and the force sensor 26 can be located in a headband/sweatband and configured to perform skin readings on the head (e.g., temples or forehead). In another embodiment, the light source 12, the optical detector 24, and the force sensor 26 can be located in a mouth guard and configured to perform gum readings. In some embodiments, the one or more processors 14 can be configured to utilize voice activation. For example, the light source 12, the optical detector 24, and the force sensor 26 can be communicatively coupled to a smart phone or a smart watch. Accordingly, in some embodiments, the system can further comprise input components such as, for example, touch screens, microphones, buttons, or the like.

### EXAMPLE 1

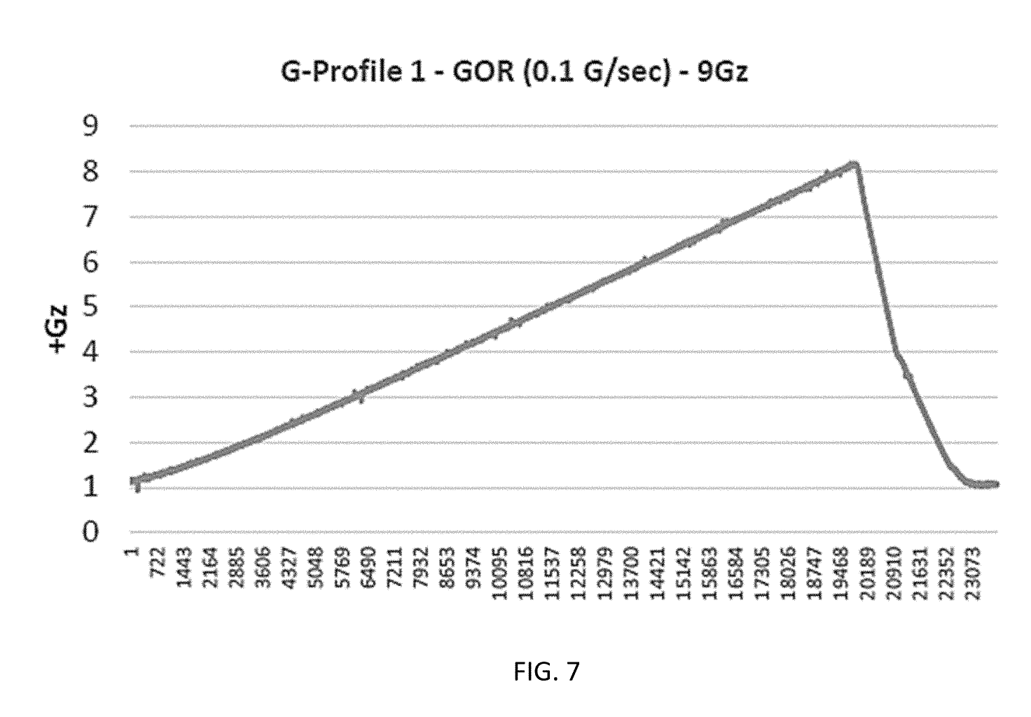

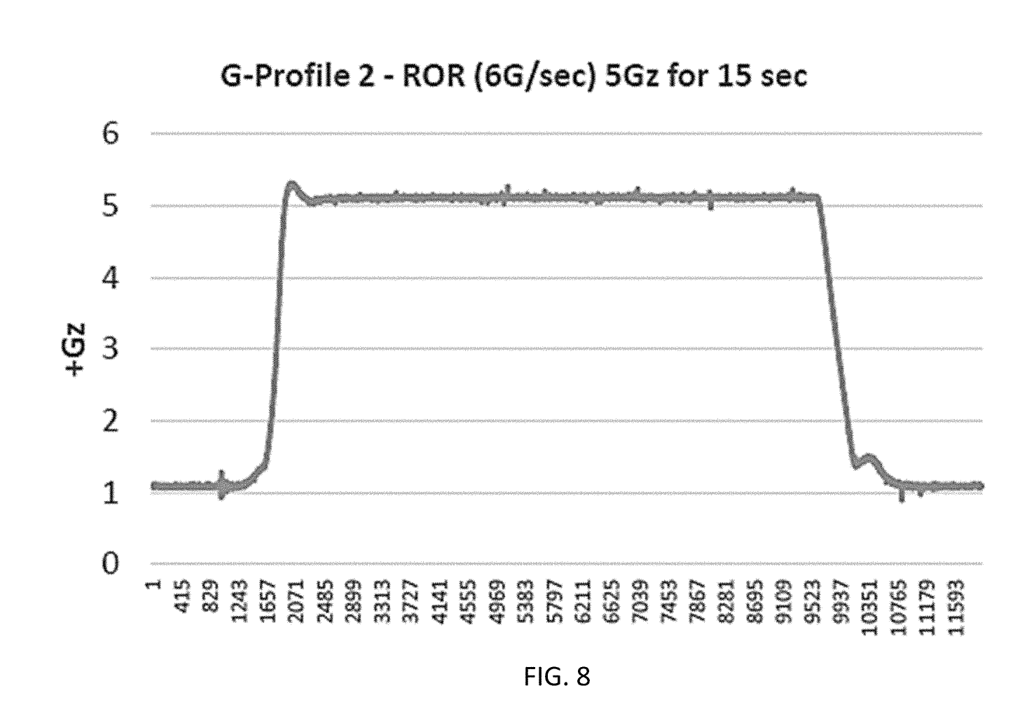

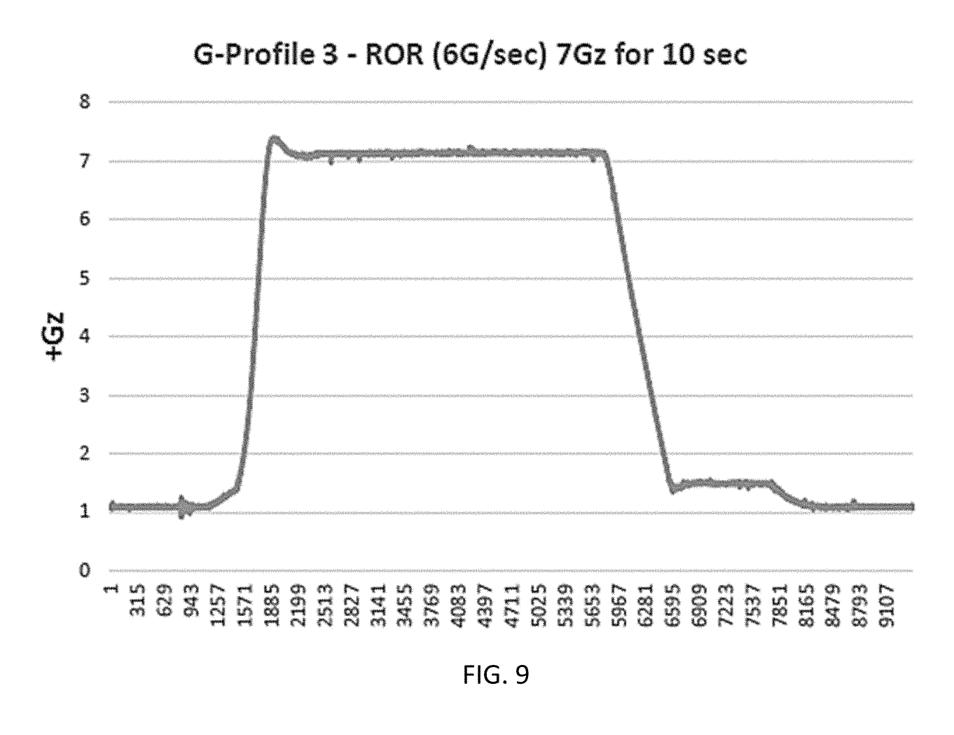

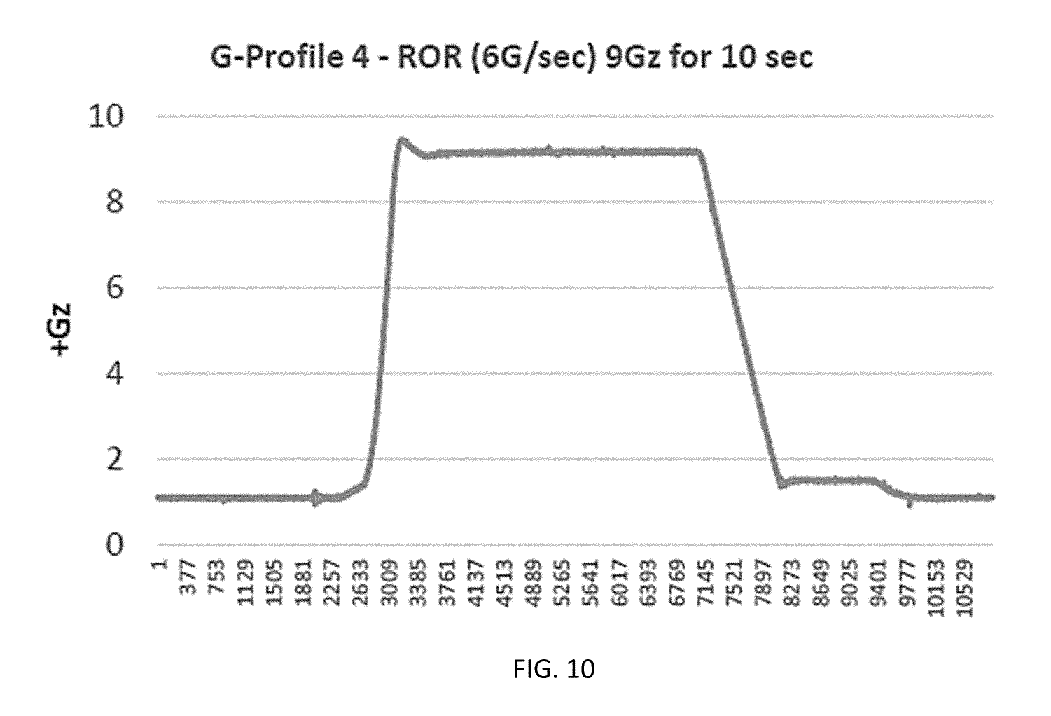

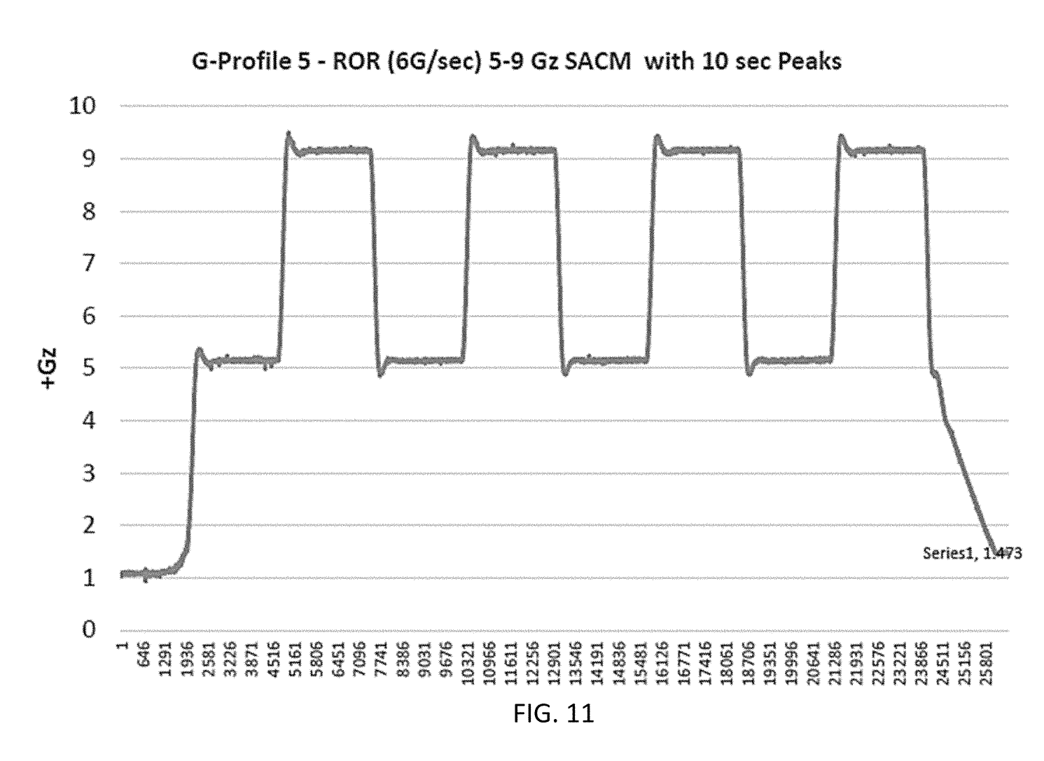

Live subjects were fitted with ear plug integrated pulse oximetry systems. The subjects were tested in a centrifuge facility, where the subjects experienced various rates of G-forces through the following G-profiles: (1) Gradual onset 0.1 G/sec onset rate to a maximum of +9 Gz (FIG. 7); (2) Rapid onset 6 G/sec onset rate to 5 Gz for 15 sec (FIG. 8); (3) Rapid onset 6 G/sec onset rate exposure to +7 Gz for 10 sec (FIG. 9); (4) Rapid onset 6 G/sec onset rate to +9 Gz for 10 sec (FIG. 10); and (5) Rapid onset 6 G/sec onset rate +5 Gz to +9 Gz simulated aerial combat maneuver (FIG. 11).

Two minutes of baseline data were acquired prior to the hypoxia exposure. The centrifuge exposure was aborted when either the acceleration profile is complete, or the subject experiences gravity-induced loss of consciousness (“GLOC”), or if the subject aborts the exposure for any reason.

Ten trained altitude test subjects from the Wyle Laboratories altitude test panel Brooks City Base participated in this evaluation. Participants experienced two sets of altitude exposures on a single test day. Altitude profiles included a sinus and ear check at 5,000 ft. followed by a hypoxia exposure to 17,500 ft. The altitude exposure was aborted when the subject aborted the exposure for any reason or when arterial blood oxygen saturation (SpO2) reached a minimum of 75%, at which time the subject was placed on 100% oxygen and altitude returned to ground level. Two minutes of baseline data was acquired prior to the hypoxia exposure.

During testing, an ear plug integrated pulse oximetry system, including a heart rate sensor, was worn by the subjects. Additionally, the subjects wore a separate, wrist-mounted heart rate sensor. The wrist-mounted heart rate sensor was purchased off the shelf, whereas the ear plug heart rate sensor was an inventive ear plug heart rate sensor as disclosed herein.

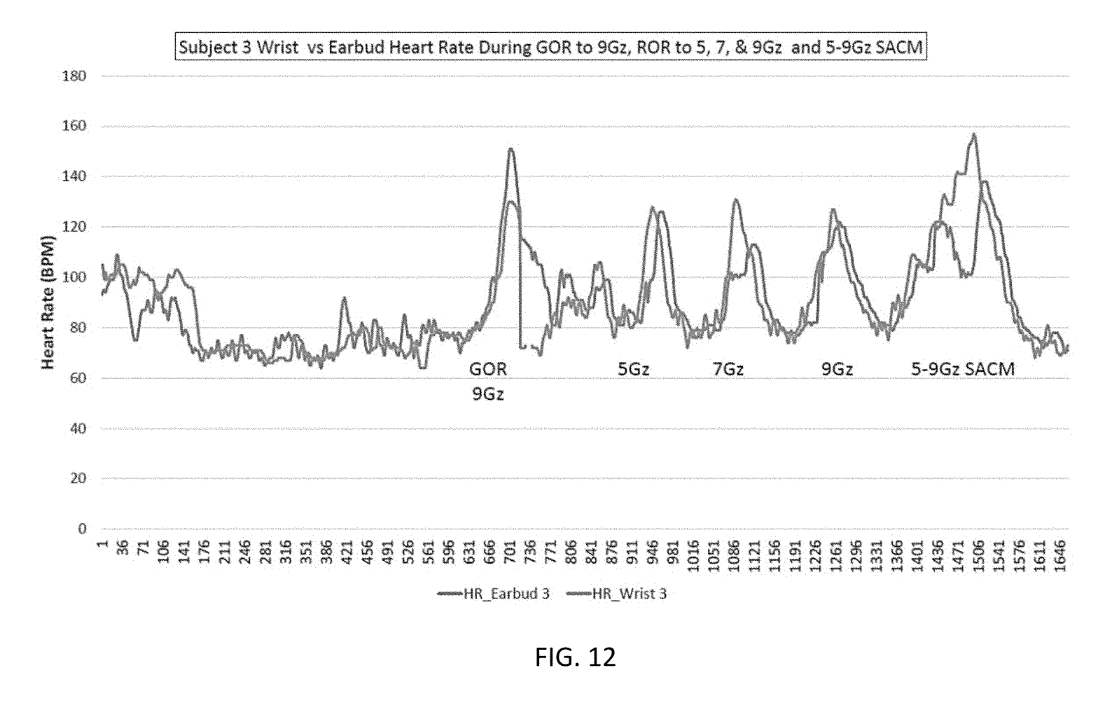

FIG. 12 illustrates a comparison of the ear plug heart rate sensor and a wrist-mounted heart rate sensor during exposure of Subject 3 to the various G-profiles.

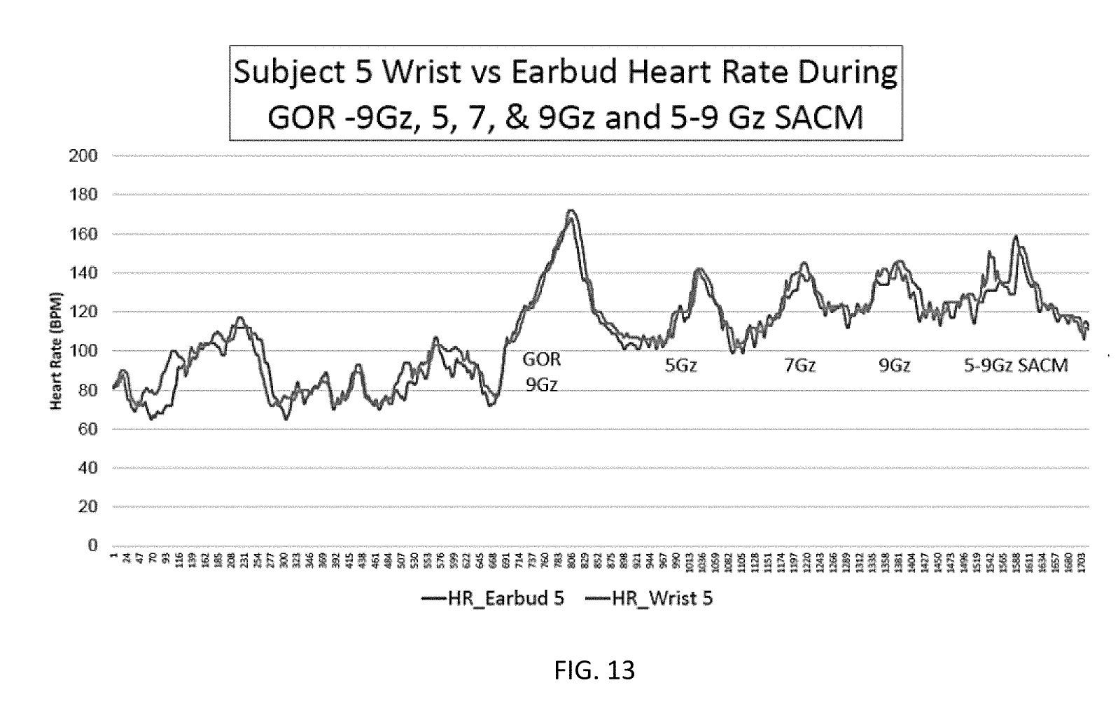

FIG. 13 illustrates a comparison of the ear plug heart rate sensor and a wrist-mounted heart rate sensor during exposure of Subject 5 to the various G-profiles.

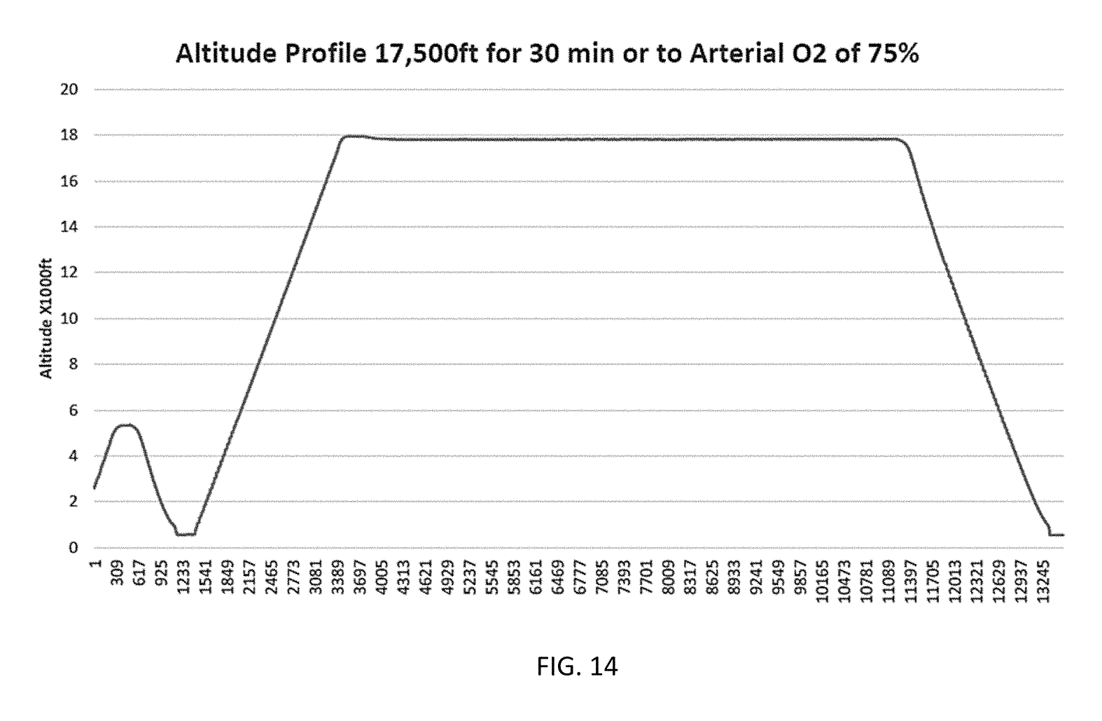

FIG. 14 illustrates the altitude profile to which the subjects were exposed.

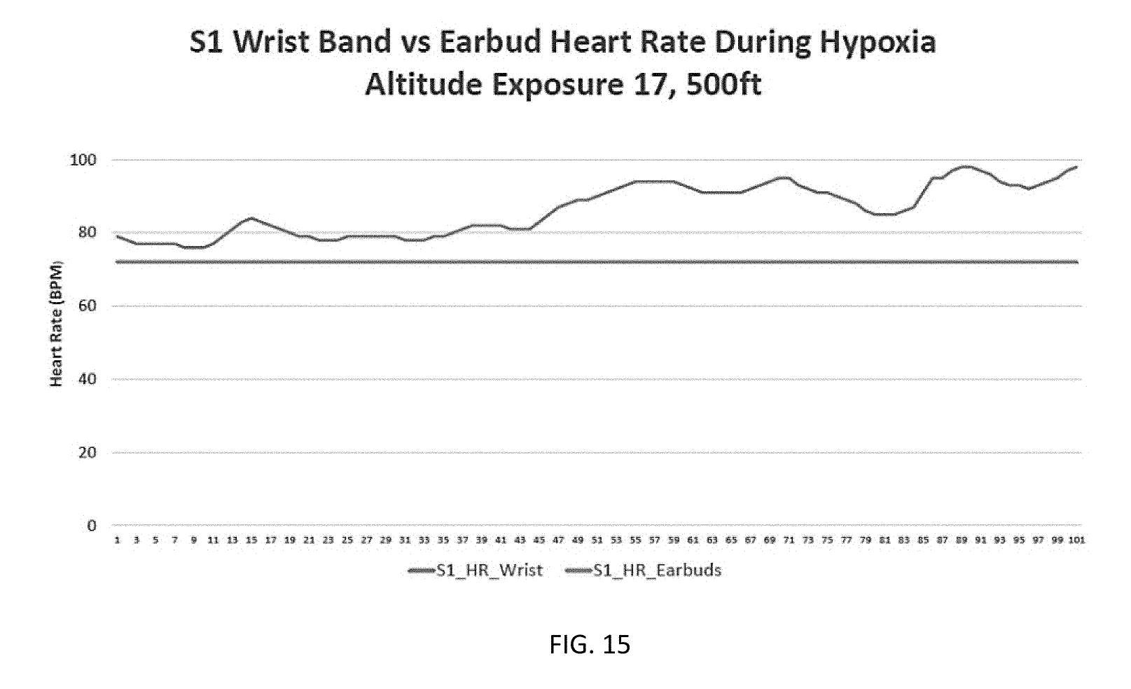

FIG. 15 illustrates a comparison of the ear plug heart rate sensor and a wrist-mounted heart rate sensor during exposure of Subject 1 to hypoxia at a simulated altitude of 17,500 ft.

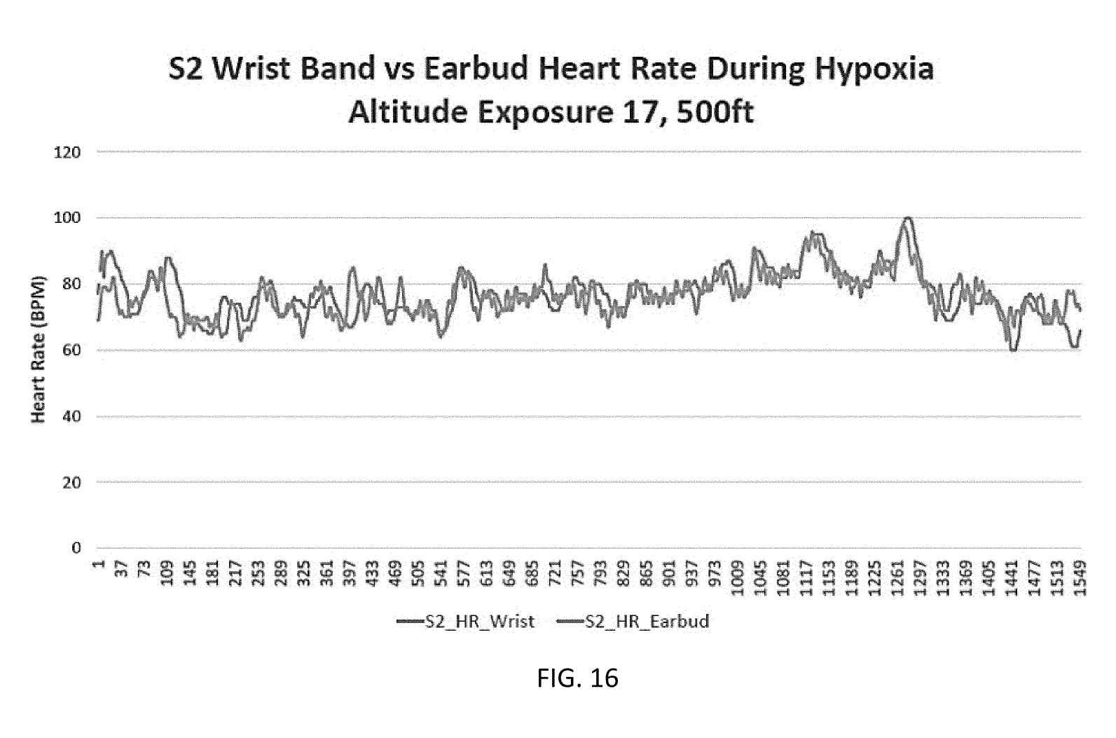

FIG. 16 illustrates a comparison of the ear plug heart rate sensor and a wrist-mounted heart rate sensor during exposure of Subject 2 to hypoxia at a simulated altitude of 17,500 ft.

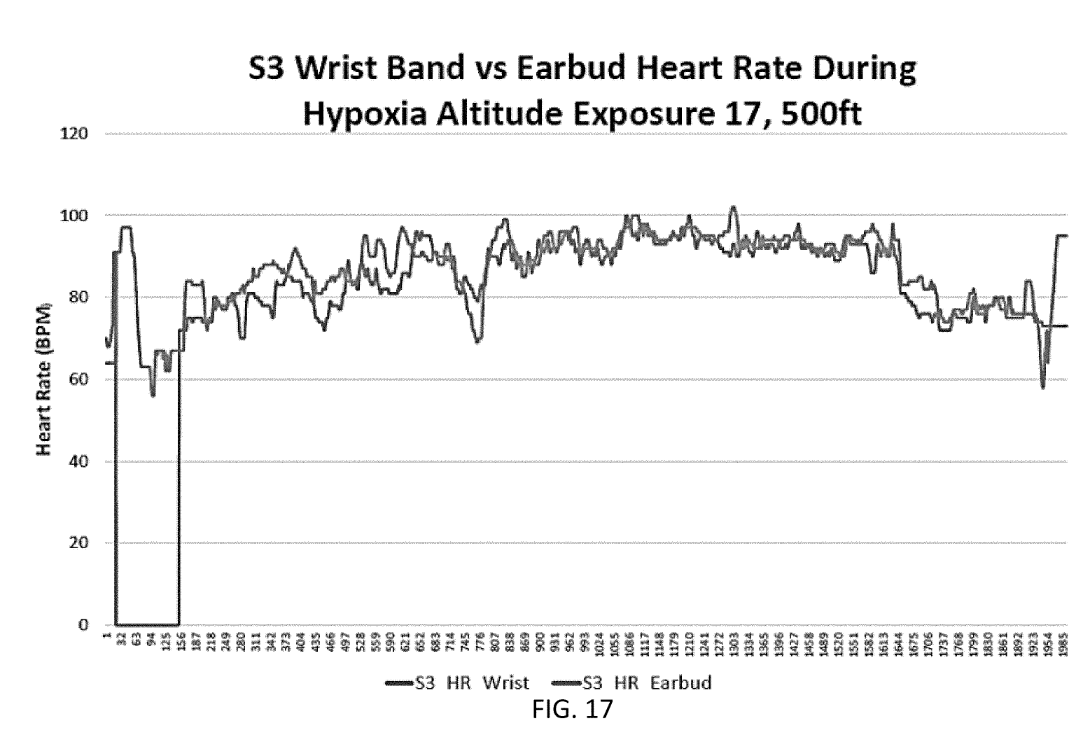

FIG. 17 illustrates a comparison of the ear plug heart rate sensor and a wrist-mounted heart rate sensor during exposure of Subject 3 to hypoxia at a simulated altitude of 17,500 ft.

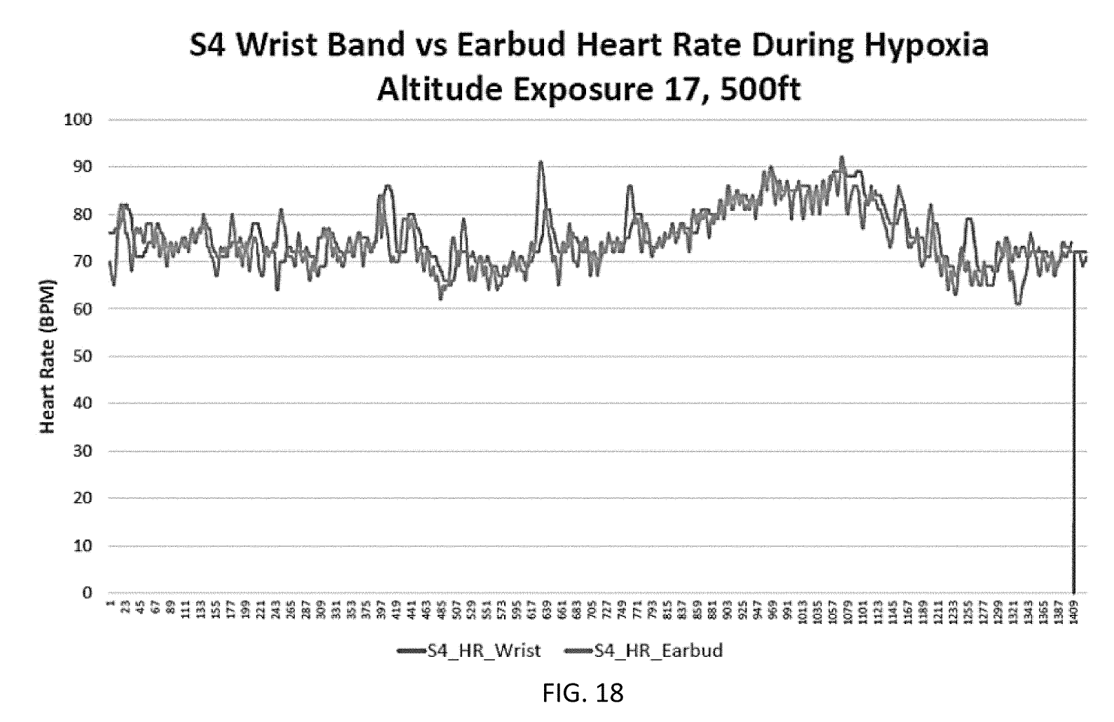

FIG. 18 illustrates a comparison of the ear plug heart rate sensor and a wrist-mounted heart rate sensor during exposure of Subject 4 to hypoxia at a simulated altitude of 17,500 ft.

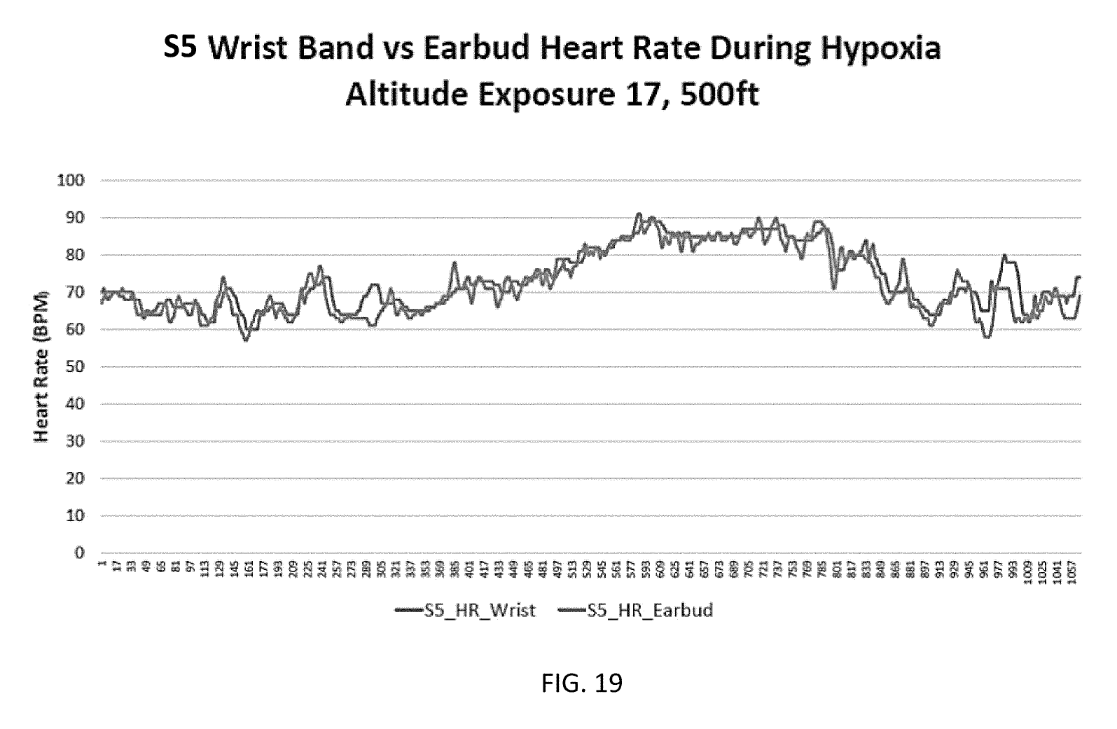

FIG. 19 illustrates a comparison of the ear plug heart rate sensor and a wrist-mounted heart rate sensor during exposure of Subject 5 to hypoxia at a simulated altitude of 17,500 ft.

As illustrated in the test results of the various subjects, the ear plug heart rate sensor performed with less error and more accuracy as compared to the wrist-mounted heart rate sensor.

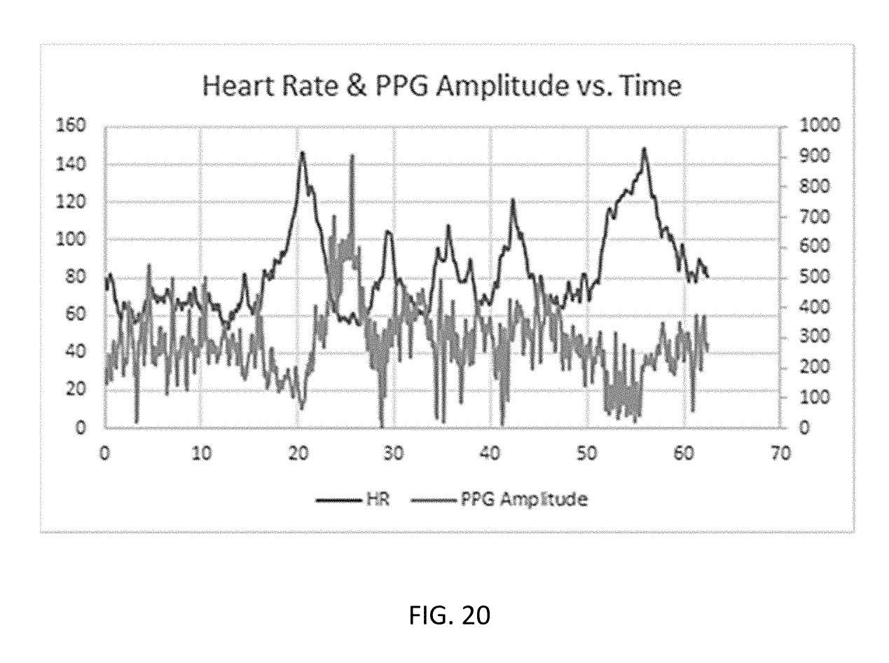

FIG. 20 illustrates a comparison of the heart rate and photoplethysmogram (“PPG”) versus time of the ear plug heart rate sensor.

It should now be understood that the embodiments described herein can be utilized as a training aid for athletes or as a monitoring device for medical professionals. For example, sports trainers can monitor participants in practice sessions or on the sidelines during games. The body metric measurements can be used to inform decisions on when rest is needed, if hydration is needed, if the athlete is overheating, or whether any other any other actionable condition exists.

Additionally, the embodiments described herein can be used to collect a data set over time for use by individuals and medical professionals, e.g., blood pressure, etc. A user can build a database of contextual, holistic body metric information without any invasive procedures, uncomfortable devices or human intermediaries. At the user's option, the database can be shared with health care professionals to diagnose and treat. Moreover, the combination of multiple sets of user data can help to develop better interventions and treatments for common problems in health and climate-controlled environments.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A method for providing body metric measurements comprising:
detecting reflected optical signals from a body of a user with an optical detector, wherein the reflected optical signals comprise red light and infrared light;
detecting a force applied to the user with a force sensor, wherein the force sensor is integrated into a wearable device; and
transforming, automatically with one or more processors, signals indicative of the reflected optical signals and the force into body metric measurements.

2. The method of claim 1, further comprising providing a light source communicatively coupled to the one or more processors.

3. The method of claim 1, wherein the optical signals comprise a first optical signal of red light having a wavelength between about 620 nm and about 750 nm.

4. The method of claim 1, wherein the optical signals comprise a second optical signal of infrared light having a wavelength between about 750 nm and about 1 mm.

5. The method of claim 1, wherein the optical detector is communicatively coupled to the one or more processors.

6. The method of claim 1, wherein the force sensor detects linear or directional motion, and wherein the linear or directional motion is correlated to impact force.

7. The method of claim 1, wherein the force sensor detects rotational motion, and wherein the rotational motion is correlated to whiplash.

8. The method of claim 1, providing a unitary device containing the one or more processors, the optical detector, and the force sensor.

9. The method of claim 1, providing multiple devices communicatively coupled with one another containing the one or more processors, the optical detector, and the force sensor.

10. The method of claim 1, providing a sensing component sized to position a sensing system chip within an ear of a user, the sensing component containing the one or more processors, the optical detector, and the force sensor.

11. A system for providing body metric measurements comprising:
a light source providing a red light and an infrared light;
an optical detector configured to receive reflected optical signals;
force sensor, wherein the force sensor is integrated into a wearable device; and
one or more processor configured to automatically transform signals indicative of the reflected optical signals and the force into body metric measurements.

12. The system of claim 11, wherein the light source is communicatively coupled to the one or more processors.

13. The system of claim 11, wherein the optical signals comprise a first optical signal of red light having a wavelength between about 620 nm and about 750 nm.

14. The system of claim 11, wherein the optical signals comprise a second optical signal of infrared light having a wavelength between about 750 nm and about 1 mm.

15. The system of claim 11, wherein the optical detector is communicatively coupled to the one or more processors.

16. The system of claim 11, wherein the force sensor detects linear or directional motion.

17. The system of claim 11, wherein the force sensor detects rotational motion.

18. The system of claim 11, further comprising a unitary device containing the one or more processors, the optical detector, and the force sensor.

19. The system of claim 11, further comprising multiple devices communicatively coupled with one another containing the one or more processors, the optical detector, and the force sensor.

20. The system of claim 11, further comprising a sensing component sized to position a sensing system chip within an ear of a user, the sensing component containing the one or more processors, the optical detector, and the force sensor.
